(19) United States
(12) Patent Application Publication
(10) Pub. No.: US 2010/0292335 A1
(43) Pub. Date: Nov. 18, 2010

(54) USE OF ORGANIC COMPOUNDS

(76) Inventors: Duncan J. Campbell, Victoria (AU); Richard Gilbert, Ontario (CA); Darren Kelly, Victoria (AU)

Correspondence Address: DILWORTH & BARRESE, LLP, 1000 WOODBURY ROAD, SUITE 405, WOODBURY, NY, 11797, US

(21) Appl. No.: 12/526,974

(22) PCT Filed: Feb. 14, 2008
(86) PCT No.: PCT/EP08/51807

§ 371 Date: Aug. 2, 2010

(30) Foreign Application Priority Data
Feb. 16, 2007 (EP) 07102590.2

(51) Int. Cl. A61K 31/165; C07C 237/22; A61P 9/10

(52) U.S. Cl. 514/616; 564/157

(57) ABSTRACT

The present invention provides methods for the prevention of, delay of progression to or the treatment of diseases modulated by an increase in tissue bradykinin levels by administering to a warm-blooded animal a therapeutically effective amount of a renin inhibitor, or a pharmaceutically acceptable salt thereof, alone or in combination with (i)) an ACE inhibitor or a pharmaceutically acceptable salt thereof, (II) an angiotensin II receptor blocker, or a pharmaceutically acceptable salt of either.

The natural enzyme renin passes from the kidneys into the blood where it effects the cleavage of angiotensinogen, releasing the decapeptide angiotensin I which is then cleaved in the lungs, the kidneys and other organs to form the octapeptide angiotensinogen II. The octapeptide increases blood pressure both directly by arterial vasoconstriction and indirectly by liberating from the adrenal glands the sodium-ion-retaining hormone aldosterone, accompanied by an increase in extracellular fluid volume. That increase can be attributed to the action of angiotensin II. Inhibitors of the enzymatic activity of renin bring about a reduction in the formation of angiotensin I. As a result a smaller amount of angiotensin II is produced. The reduced concentration of that active peptide hormone is the direct cause of, e.g., the hypotensive effect of renin inhibitors.

Further evaluations have revealed that renin inhibitors may also be employed for a broader range of therapeutic indications.

It has now surprisingly been found that renin inhibitors, in particular aliskiren, may be employed for prevention of, delay progression to overt to, or the treatment of diseases of the heart, in particular ischaemic heart disease or ischaemic heart damage, due to its ability to increase tissue bradykinin levels. Even more surprisingly, this effect has been shown so far only with ACE inhibitors in high doses (see Hypertension, May 1995; 25: 1014-1020) but not other drugs such as NEP inhibitors (see J. Pharmacol. Experimental Therapeutics, 1999, vol. 289, No. 1, 295-303), whereby this effect of renin inhibitors on bradykinin levels is very robust.

Accordingly, the present invention relates to a method for the prevention of, delay progression to overt to, or the treatment of diseases modulated by an increase in tissue bradykinin levels which method comprises administering to a warm-blooded animal a therapeutically effective amount of a renin inhibitor, or a pharmaceutically acceptable salt thereof.

Diseases modulated by an increase in tissue bradykinin levels as used herein refers in particular to diseases modulated by an increase in tissue bradykinin levels in the heart. These diseases are treated by, their progression is delayed by or they can be prevented by an increase in tissue bradykinin levels. These are diseases of the heart and can include effects on the metabolism of the heart and the ability of the cardiomyocyte to withstand ischaemic stress. Typically, the diseases include ischaemic heart disease and ischaemic heart damage. Although the effects of kinins on the heart are not completely understood, they are well documented and are clearly related to therapeutic benefits (see e.g. Spillmann F, Van Linthout S, Schultheiss H P, Tschope C. Cardioprotective mechanisms of the kallikrein-kinin system in diabetic cardiopathy. Curr Opin Nephrol Hypertens 2006; 15:22-29; Park S S, Zhao H, Mueller R A, Xu Z. Bradykinin prevents reperfusion injury by targeting mitochondrial permeability transition pore through glycogen synthase kinase 3beta. J Mol Cell Cardiol 2006; 40:708-716; Koch M, Spillmann F, Dendorfer A, Westermann D, Altmann C, Sahabi M, Linthout S V, Bader M, Walther T, Schultheiss H P, Tschope C. Cardiac function and remodeling is attenuated in transgenic rats expressing the human kallikrein-1 gene after myocardial infarction. Eur J Pharmacol 2006; 550:143-148; Griol-Charhbili V, Messadi-Laribi E, Bascands J L, Heudes D, Meneton P, Giudicelli J F, Alhenc-Gelas F, Richer C. Role of tissue kallikrein in the cardioprotective effects of ischemic and pharmacological preconditioning in myocardial ischemia. Faseb J2005; 19:1172-1174). The potential role of bradykinin in promoting angiogenesis has also been reported (see Peter Gohlke, Ingo Kuwer, Angela Schnell, Kerstin Amann, Gerhard Mall, and Thomas Unger. Blockade of Bradykinin B2 Receptors Prevents the Increase in Capillary Density Induced by Chronic Angiotensin-Converting Enzyme Inhibitor Treatment in Stroke-Prone Spontaneously Hypertensive Rats. Hypertension, January 1997; 29: 478-482). Promotion of angiogenesis in the heart may be of particular benefit for ischaemic heart disease.

Furthermore, it has now been shown that a combination of a renin inhibitor with an (i) ACE inhibitor or (ii) an angiotensin II receptor blocker confers added or synergistic therapeutic effects over each monotherapy component alone. The preferred combination partner is an ACE inhibitor for this indication.

Accordingly, the present invention further relates to a method for the prevention of, delay progression to overt to, or the treatment of diseases modulated by an increase in tissue bradykinin levels which method comprises administering to a warm-blooded animal a therapeutically effective amount of a combination of a renin inhibitor, or a pharmaceutically acceptable salt thereof, with
(i) an ACE inhibitor, or a pharmaceutically acceptable salt thereof; or
(II) an angiotensin II receptor blocker, or a pharmaceutically acceptable salt thereof.

Other objects, features, advantages and aspects of the present invention will become apparent to those skilled in the art from the following description and appended claims. It should be understood, however, that the following description, appended claims, and specific examples, while indicating preferred embodiments of the invention, are given by way of illustration only. Various changes and modifications within the spirit and scope of the disclosed invention will become readily apparent to those skilled in the art from reading the following. Abbreviations are those generally known in the art.

Listed below are the definitions of various terms used herein to describe certain aspects of the present invention. However, the definitions and abbreviations thereof used herein are those generally known in the art and apply to the terms as they are used throughout the specification unless they are otherwise limited in specific instances.

The term “prevention” refers to prophylactic administration to healthy patients to prevent the development of the conditions mentioned herein. Moreover, the term “prevention” means prophylactic administration to patients being in a pre-stage of the conditions to be treated.

The term “delay progression to overt to”, as used herein, refers to administration to patients being in a pre-stage of the condition to be treated in which patients with a pre-form of the corresponding condition is diagnosed.

The term “treatment” is understood the management and care of a patient for the purpose of combating the disease, condition or disorder.

The term “therapeutically effective amount” refers to an amount of a drug or a therapeutic agent that will elicit the desired biological or medical response of a tissue, system or an animal (including man) that is being sought by a researcher or clinician.

The term “synergistic”, as used herein, means that the effect achieved with the methods, combinations and pharmaceutical compositions of the present invention is greater than the sum of the effects that result from individual methods and compositions comprising the active ingredients of this invention separately.

The term “warm-blooded animal or patient” are used interchangeably herein and include, but are not limited to, humans, dogs, cats, horses, pigs, cows, monkeys, rabbits, mice and laboratory animals. The preferred mammals are humans.

The term “pharmaceutically acceptable salt” refers to a non-toxic salt commonly used in the pharmaceutical industry which may be prepared according to methods well-known in the art.

The term “combination” of a renin inhibitor, in particular, aliskiren, and an ACE inhibitor or an angiotensin II receptor blocker, or in each case, a pharmaceutically acceptable salt thereof, means that the components can be administered together as a pharmaceutical composition or as part of the same, unitary dosage form. A combination also includes administering a renin inhibitor, in particular, aliskiren, or a pharmaceutically acceptable salt thereof, and an ACE inhibitor or an angiotensin II receptor blocker, or in each case, a pharmaceutically acceptable salt thereof, each separately but as part of the same therapeutic regimen. The components, if administered separately, need not necessarily be administered at essentially the same time, although they can if so desired. Thus, a combination also refers, for example, administering a renin inhibitor, in particular, aliskiren, or a pharmaceutically acceptable salt thereof, and an ACE inhibitor or an angiotensin II receptor blocker, or in each case, a pharmaceutically acceptable salt thereof, as separate dosages or dosage forms, but at the same time. A combination also includes separate administration at different times and in any order.

The renin inhibitors to which the present invention applies are any of those having renin inhibitory activity in vivo and, therefore, pharmaceutical utility, e.g., as therapeutic agents for the prevention of, delay progression to overt to, or the treatment of diseases modulated by an increase in tissue bradykinin levels. In particular, the present invention relates to renin inhibitors disclosed in U.S. Pat. No. 5,559,111; No. 6,197,959 and No. 6,376,672.

Renin inhibitors include compounds having different structural features. For example, mention may be made of compounds which are selected from the group consisting of ditekiren (chemical name: [1S-[1R*,2R*,4R*(1R*,2R*)]]-1-[(1,1-dimethylethoxy)carbonyl]-L-proly I-L-phenylalanyl-N-[2-hydroxy-5-methyl-1-(2-methylpropyl)-4-[[[2-methyl-1-[[(2-pyridinylmrthyl)amino]carbonyl]butyl]amino]carbonyl]hexyl]-N-alfa-methyl-L-histidinamide); terlakiren (chemical name: [R—(R*,S*)]-N-(4-morpholinylcarbonyl)-L-phenylalanyl-N-β-(cyclohexy (methyl)-2-hydroxy-3-(1-methylethoxy)-3-oxopropyl]-S-methyl-L-cysteineamide); and zankiren (chemical name: [1S-[1R*[R*(R*)],2S*,3R*]]-N-[1-(cyclohexylmethyl)-2,3-dihydroxy-5-methylhexyl]-alfa[[2-[[(4-methyl-1-piperazinyl)sulfonyl]methyl]-1-oxo-3-phenylpropyl]amino]-4-thiazolepropanamide), preferably, in each case, the hydrochloride salt thereof.

Preferred renin inhibitor of the present invention include RO 66-1132 and RO 66-1168 of formulae (I) and (II)

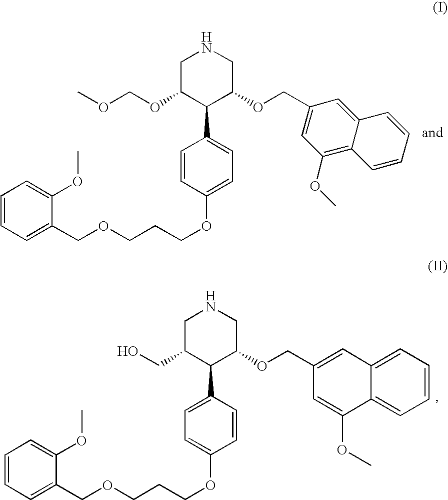

respectively, or in each case, a pharmaceutically acceptable salt thereof.

In particular, the present invention relates to a renin inhibitor which is a δ-amino-γ-hydroxy-ω-aryl-alkanoic acid amide derivative of the formula

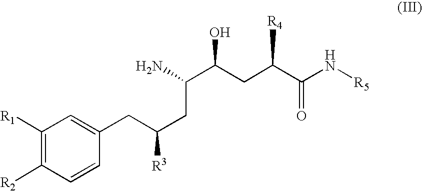

wherein R₁ is halogen, C₁₋₆halogenalkyl, C₁₋₆alkoxy-C₁₋₆alkyloxy or C₁₋₆alkoxy-C₁₋₆alkyl; R₂ is halogen, C₁₋₄alkyl or C₁₋₄alkoxy; R₃ and R₄ are independently branched C₃₋₆alkyl; and R₅ is cycloalkyl, C₁₋₆alkyl, C₁₋₆hydroxyalkyl, C₁₋₆alkoxy-C₁₋₆alkyl, C₁₋₆alkanoyloxy-C₁₋₆alkyl, C₁₋₆-aminoalkyl, C₁₋₆alkylamino-C₁₋₆alkyl, C₁₋₆dialkylamino-C₁₋₆alkyl, C₁₋₆alkanoylamino-C₁₋₆alkyl, HO(O)C-C₁₋₆alkyl, C₁₋₆alkyl-O—(O)C-C₁₋₆alkyl, H₂N—C(O)—C₁₋₆alkyl, C₁₋₆alkyl-HN—C(O)—C₁₋₆alkyl or (C₁₋₆alkyl)₂N—C(O)—C₁₋₆alkyl; or a pharmaceutically acceptable salt thereof.

As an alkyl, R₁ may be linear or branched and preferably comprise 1 to 6 C atoms, especially 1 or 4 C atoms. Examples are methyl, ethyl, n- and i-propyl, n-, i- and t-butyl, pentyl and hexyl.

As a halogenalkyl, R₁ may be linear or branched and preferably comprise 1 to 4 C atoms, especially 1 or 2 C atoms. Examples are fluoromethyl, difluoromethyl, trifluoromethyl, chloromethyl, dichloromethyl, trichloromethyl, 2-chloroethyl and 2,2,2-trifluoroethyl.

As an alkoxy, R₁ and R₂ may be linear or branched and preferably comprise 1 to 4 C atoms. Examples are methoxy, ethoxy, n- and i-propyloxy, n-, i- and t-butyloxy, pentyloxy and hexyloxy.

As an alkoxyalkyl, R₁ may be linear or branched. The alkoxy group preferably comprises 1 to 4 and especially 1 or 2 C atoms, and the alkyl group preferably comprises 1 to 4 C atoms. Examples are methoxymethyl, 2-methoxyethyl, 3-methoxypropyl, 4-methoxybutyl, 5-methoxypentyl, 6-methoxyhexyl, ethoxymethyl, 2ethoxyethyl, 3-ethoxypropyl, 4-ethoxybutyl, 5-ethoxypentyl, 6-ethoxyhexyl, propyloxymethyl, butyloxymethyl, 2-propyloxyethyl and 2-butyloxyethyl.

As a C₁₋₆alkoxy-C₁₋₆alkyloxy, R₁ may be linear or branched. The alkoxy group preferably comprises 1 to 4 and especially 1 or 2 C atoms, and the alkyloxy group preferably comprises 1 to 4 C atoms. Examples are methoxymethyloxy, 2-methoxyethyloxy, 3-methoxypropyloxy, 4-methoxybutyloxy, 5-methoxypentyloxy, 6-methoxyhexyloxy, ethoxymethyloxy, 2-ethoxyethyloxy, 3-ethoxypropyloxy, 4-ethoxybutyloxy, 5-ethoxypentyloxy, 6-ethoxyhexyloxy, propyloxymethyloxy, butyloxymethyloxy, 2-propyloxyethyloxy and 2-butyloxyethyloxy.

In a preferred embodiment, R₁ is methoxy- or ethoxy-C₁₋₄alkyloxy, and R₂ is preferably methoxy or ethoxy. Particularly preferred are compounds of formula (III), wherein R₁ is 3-methoxypropyloxy and R₂ is methoxy.

As a branched alkyl, R₃ and R₄ preferably comprise 3 to 6 C atoms. Examples are i-propyl, i- and t-butyl, and branched isomers of pentyl and hexyl. In a preferred embodiment, R₃ and R₄ in compounds of formula (III) are in each case i-propyl.

As a cycloalkyl, R₅ may preferably comprise 3 to 8 ring-carbon atoms, 3 or 5 being especially preferred. Some examples are cyclopropyl, cyclobutyl, cyclopentyl, cyclohexyl and cyclooctyl. The cycloalkyl may optionally be substituted by one or more substituents, such as alkyl, halo, oxo, hydroxy, alkoxy, amino, alkylamino, dialkylamino, thiol, alkylthio, nitro, cyano, heterocyclyl and the like.

As an alkyl, R₅ may be linear or branched in the form of alkyl and preferably comprise 1 to 6 C atoms. Examples of alkyl are listed herein above. Methyl, ethyl, n- and i-propyl, n-, i- and t-butyl are preferred.

As a C₁₋₆hydroxyalkyl, R₅ may be linear or branched and preferably comprise 2 to 6 C atoms. Some examples are 2-hydroxyethyl, 2-hydroxypropyl, 3-hydroxypropyl, 2-, 3- or 4-hydroxybutyl, hydroxypentyl and hydroxyhexyl.

As a C₁₋₆alkoxy-C₁₋₆alkyl, R₅ may be linear or branched. The alkoxy group preferably comprises 1 to 4 C atoms and the alkyl group preferably 2 to 4 C atoms. Some examples are 2-methoxyethyl, 2-methoxypropyl, 3-methoxypropyl, 2-, 3- or 4-methoxybutyl, 2-ethoxyethyl, 2-ethoxypropyl, 3-ethoxypropyl, and 2-, 3- or 4-ethoxybutyl.

As a C₁₋₆alkanoyloxy-C₁₋₆alkyl, R₅ may be linear or branched. The alkanoyloxy group preferably comprises 1 to 4 C atoms and the alkyl group preferably 2 to 4 C atoms. Some examples are formyloxymethyl, formyloxyethyl, acetyloxyethyl, propionyloxyethyl and butyroyloxyethyl.

As a C₁₋₆aminoalkyl, R₅ may be linear or branched and preferably comprise 2 to 4 C atoms. Some examples are 2-aminoethyl, 2- or 3-aminopropyl and 2-, 3- or 4-aminobutyl.

As C₁₋₆alkylamino-C₁₋₆alkyl and C₁₋₆dialkylamino-C₁₋₆alkyl, R₅ may be linear or branched. The alkylamino group preferably comprises C₁₋₄alkyl groups and the alkyl group has preferably 2 to 4 C atoms. Some examples are 2-methylaminoethyl, 2-dimethylaminoethyl, 2-ethylaminoethyl, 2-ethylaminoethyl, 3-methylaminopropyl, 3-dimethylaminopropyl, 4-methylaminobutyl and 4-dimethylaminobutyl.

As a HO(O)C-C₁₋₆alkyl, R₅ may be linear or branched and the alkyl group preferably comprises 2 to 4 C atoms. Some examples are carboxymethyl, carboxyethyl, carboxypropyl and carboxybutyl.

As a C₁₋₆alkyl-O—(O)C-C₁₋₆alkyl, R₅ may be linear or branched, and the alkyl groups preferably comprise independently of one another 1 to 4 C atoms. Some examples are methoxycarbonylmethyl, 2-methoxycarbonylethyl, 3-methoxycarbonylpropyl, 4-methoxy-carbonylbutyl, ethoxycarbonylmethyl, 2-ethoxycarbonylethyl, 3-ethoxycarbonylpropyl, and 4-ethoxycarbonylbutyl.

As a H₂N—C(O)—C₁₋₆alkyl, R₅ may be linear or branched, and the alkyl group preferably comprises 2 to 6 C atoms. Some examples are carbamidomethyl, 2-carbamidoethyl, 2-carbamido-2,2-dimethylethyl, 2- or 3-carbamidopropyl, 2-, 3- or 4-carbamidobutyl, 3-carbamido-2-methylpropyl, 3-carbamido-1,2-dimethylpropyl, 3-carbamido-3-ethylpropyl, 3-carbamido-2,2-dimethylpropyl, 2-, 3-, 4- or 5-carbamidopentyl, 4-carbamido-3,3- or -2,2-dimethylbutyl.

Accordingly, preferred are δ-amino-γ-hydroxy-ω-aryl-alkanoic acid amide derivatives of formula (III) having the formula

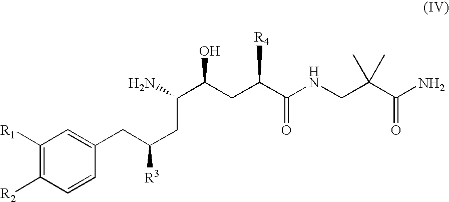

wherein R₁ is 3-methoxypropyloxy; R₂ is methoxy; and R₃ and R₄ are isopropyl; or a pharmaceutically acceptable salt thereof; chemically defined as 2(S),4(S),5(S),7(S)-N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide, also known as aliskiren.

The term “aliskiren”, if not defined specifically, is to be understood both as the free base and as a salt thereof, especially a pharmaceutically acceptable salt thereof, most preferably a hemi-fumarate thereof.

Angiotensin II receptor blockers are understood to be those active agents that bind to the AT₁-receptor subtype of angiotensin II receptor but do not result in activation of the receptor. As a consequence of the blockade of the AT₁ receptor, these antagonists can, e.g., be employed as antihypertensive agents.

Suitable angiotensin II receptor blockers which may be employed in the combination of the present invention include AT₁ receptor antagonists having differing structural features, preferred are those with the non-peptidic structures. For example, mention may be made of the compounds that are selected from the group consisting of valsartan (EP 443983), losartan (EP253310), candesartan (EP 459136), eprosartan (EP 403159), irbesartan (EP 454511), olmesartan (EP 503785), tasosartan (EP539086), telmisartan (EP 522314), the compound with the designation E-4177 of the formula

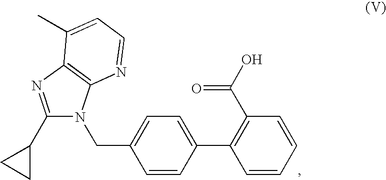

the compound with the designation SC-52458 of the following formula

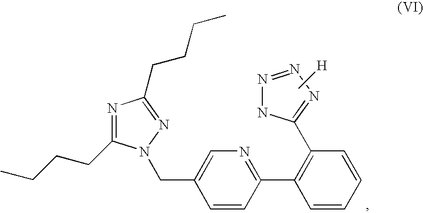

and the compound with the designation the compound ZD-8731 of the formula

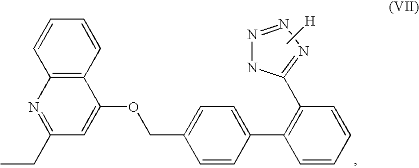

or, in each case, a pharmaceutically acceptable salt thereof.

Preferred AT₁-receptor antagonists are those agents that have reach the market, most preferred is valsartan, or a pharmaceutically acceptable salt thereof.

The interruption of the enzymatic degradation of angiotensin Ito angiotensin II with ACE inhibitors is a successful variant for the regulation of blood pressure and thus also makes available a therapeutic method for the treatment of hypertension.

A suitable ACE inhibitor to be employed in the combination of the present invention is, e.g., a compound selected from the group consisting alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril, and trandolapril, or in each case, a pharmaceutically acceptable salt thereof.

Preferred ACE inhibitors are those agents that have been marketed, most preferred are benazepril and enalapril.

Preferably, a combination according to the present invention comprises a renin inhibitor, e.g., aliskiren, especially in the form of the hemi-fumarate salt thereof, and an ACE inhibitor, e.g., benazepril or enalapril, or an angiotensin II receptor blocker, e.g., valsartan, or in each case, a pharmaceutically acceptable salt thereof.

Most preferred is a combination according to the present invention comprising aliskiren, especially in the form of the hemi-fumarate salt thereof, and valsartan, or a pharmaceutically acceptable salt thereof.

As referred herein above, the compounds to be combined may be present as their pharmaceutically acceptable salts. If these compounds have, e.g., at least one basic center such as an amino group, they can form acid addition salts thereof. Similarly, the compounds having at least one acid group (for example COOH) can form salts with bases.

Corresponding internal salts may furthermore be formed, if a compound comprises, e.g., both a carboxy and an amino group.

The corresponding active ingredients or a pharmaceutically acceptable salts may also be used in form of a solvate, such as a hydrate or including other solvents used, e.g., in their crystallization.

Furthermore, the present invention provides pharmaceutical compositions comprising a renin inhibitor, or a pharmaceutically acceptable salt thereof, preferably aliskiren in the form of the hemi-fumarate salt thereof, and a pharmaceutically acceptable carrier, for the prevention of, delay progression to overt to, or the treatment of diastolic dysfunction or diastolic heart failure.

In another aspect, the present invention further provides pharmaceutical compositions comprising a renin inhibitor, or a pharmaceutically acceptable salt thereof, preferably aliskiren in the form of the hemi-fumarate salt thereof, in combination with
(i) an ACE inhibitor, preferably benazepril or enalapril, or in each case, a pharmaceutically acceptable salt thereof; or
(ii) an angiotensin II receptor blocker, preferably valsartan, or a pharmaceutically acceptable salt thereof;
   and a pharmaceutically acceptable carrier; for the prevention of, delay progression to overt to, or the treatment of diseases modulated by an increase in tissue bradykinin levels.

As disclosed herein above, a renin inhibitor, in particular, aliskiren, preferably in the form of the hemi-fumarate salt thereof, alone or in combination with an ACE inhibitor, e.g., benazepril or enalapril, or an angiotensin II receptor blocker, e.g., valsartan, or in each case, a pharmaceutically acceptable salt thereof, may be co-administered as a pharmaceutical composition. The components may be administered together in any conventional dosage form, usually also together with a pharmaceutically acceptable carrier or diluent.

The pharmaceutical compositions according to the invention are those suitable for enteral, such as oral or rectal, transdermal and parenteral administration to mammals, including man. For oral administration the pharmaceutical composition comprising a renin inhibitor, in particular, aliskiren, preferably in the form of the hemi-fumarate salt thereof, alone or in combination with an ACE inhibitor, e.g., benazepril or enalapril, or an angiotensin II receptor blocker, e.g., valsartan, or in each case, a pharmaceutically acceptable salt thereof, can take the form of solutions, suspensions, tablets, pills, capsules, powders, microemulsions, unit dose packets and the like. Preferred are tablets and gelatin capsules comprising the active ingredient together with: a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbants, colorants, flavors and sweeteners. Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-90%, preferably about 1-80%, of the active ingredient.

The dosage of the active ingredients can depend on a variety of factors, such as mode of administration, homeothermic species, age and/or individual condition.

Preferred dosages for the active ingredients of the pharmaceutical combinations according to the present invention are therapeutically effective dosages, especially those which are commercially available.

Normally, in the case of oral administration, an approximate daily dose of from about 1 mg to about 360 mg is to be estimated, e.g., for a patient of approximately 75 kg in weight.

For example, the doses of aliskiren to be administered to warm-blooded animals, including man, of approximately 75 kg body weight, especially the doses effective for the inhibition of renin activity, e.g., in lowering blood pressure, are from about 3 mg to about 3 g, preferably from about 10 mg to about 1 g, e.g., from 20 to 200 mg/person/day, divided preferably into 1 to 4 single doses which may, e.g., be of the same size. Usually, children receive about half of the adult dose. The dose necessary for each individual can be monitored, e.g., by measuring the serum concentration of the active ingredient, and adjusted to an optimum level. Single doses comprise, e.g., 75 mg, 150 mg or 300 mg per adult patient.

In case of ACE inhibitors, preferred dosage unit forms of ACE inhibitors are, for example, tablets or capsules comprising e.g. from about 5 mg to about 20 mg, preferably 5 mg, 10 mg, 20 mg or 40 mg, of benazepril; from about 6.5 mg to 100 mg, preferably 6.25 mg, 12.5 mg, 25 mg, 50 mg, 75 mg or 100 mg, of captopril; from about 2.5 mg to about 20 mg, preferably 2.5 mg, 5 mg, 10 mg or 20 mg, of enalapril; from about 10 mg to about 20 mg, preferably 10 mg or 20 mg, of fosinopril; from about 2.5 mg to about 4 mg, preferably 2 mg or 4 mg, of perindopril; from about 5 mg to about 20 mg, preferably 5 mg, 10 mg or 20 mg, of quinapril; or from about 1.25 mg to about 5 mg, preferably 1.25 mg, 2.5 mg, or 5 mg, of ramipril. Preferred is t.i.d. administration.

Angiotensin II receptor blockers, e.g., valsartan, are supplied in the form of a suitable dosage unit form, e.g., a capsule or tablet, and comprising a therapeutically effective amount of an angiotensin II receptor blocker, e.g., from about 20 to about 320 mg of valsartan, which may be applied to patients. The application of the active ingredient may occur up to three times a day, starting, e.g., with a daily dose of 20 mg or 40 mg of an angiotensin II receptor blocker, e.g., valsartan, increasing via 80 mg daily and further to 160 mg daily, and finally up to 320 mg daily. Preferably, an angiotensin II receptor blocker, e.g., valsartan is applied once a day or twice a day with a dose of 80 mg or 160 mg, respectively, each. Corresponding doses may be taken, e.g., in the morning, at mid-day or in the evening.

The above doses encompass a therapeutically effective amount of the active ingredients of the present invention.

Since the present invention relates to methods for the prevention, delay progression to overt to, or the treatment with a combination of compounds which may be administered separately, the invention also relates to combining separate pharmaceutical compositions in a kit form. The kit may comprise, e.g., two separate pharmaceutical compositions: (1) a composition comprising a renin inhibitor, in particular, aliskiren, or a pharmaceutically acceptable salt thereof, and a pharmaceutically acceptable carrier or diluent; and (2) a composition comprising an ACE inhibitor, e.g., benazepril or enalapril, or an angiotensin II receptor blocker, e.g., valsartan, or in each case, a pharmaceutically acceptable salt thereof, and a pharmaceutically acceptable carrier or diluent. The amounts of (1) and (2) are such that, when co-administered separately a beneficial therapeutic effect(s) is achieved. The kit comprises a container for containing the separate compositions such as a divided bottle or a divided foil packet, wherein each compartment contains a plurality of dosage forms (e.g., tablets) comprising, e.g., (1) or (2). Alternatively, rather than separating the active ingredient-containing dosage forms, the kit may contain separate compartments each of which contains a whole dosage which in turn comprises separate dosage forms. An example of this type of kit is a blister pack wherein each individual blister contains two (or more) tablets, one (or more) tablet(s) comprising a pharmaceutical composition (1), and the second (or more) tablet(s) comprising a pharmaceutical composition (2). Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician. In the case of the instant invention a kit therefore comprises:

(1) a therapeutically effective amount of a composition comprising a renin inhibitor, in particular, aliskiren, preferably in the form of the hemi-fumarate salt thereof, and a pharmaceutically acceptable carrier or diluent, in a first dosage form;

(2) a composition comprising an ACE inhibitor, e.g., benazepril or enalapril, or an angiotensin II receptor blocker, e.g., valsartan, or in each case, a pharmaceutically acceptable salt thereof, in an amount such that, following administration, a beneficial therapeutic effect(s) is achieved, and a pharmaceutically acceptable carrier or diluent, in a second dosage form; and

(3) a container for containing said first and second dosage forms.

The action of a renin inhibitor, e.g., aliskiren, may be demonstrated inter alia experimentally by means of in vitro tests, the reduction in the formation of angiotensin I being measured in various systems (human plasma, purified human renin together with synthetic or natural renin substrate).

Since renin displays species specificity for its substrate, human renin inhibitors cannot be efficiently tested in conventional in vivo animal models. To circumvent this problem, transgenic rats have been developed harboring either the human renin or the human angiotensinogen genes. Human renin does not effectively cleave rat angiotensinogen and similarly, rat renin cleaves human angiotensinogen poorly. Consequently, the single transgenic rats (i.e., transgenic for either human angiotensinogen or renin) are normotensive. However, when crossbred, the double transgenic (dTGR) offspring develop, e.g., hypertension and diastolic dysfunction, and do not live beyond the 7^{th} or 8^{th} week of age.

A renin inhibitor, e.g., aliskiren, or a pharmaceutically acceptable salt thereof, alone or in combination with an ACE inhibitor, e.g., benazepril or enalapril, or an angiotensin II receptor blocker, e.g., valsartan, or in each case, a pharmaceutically acceptable salt thereof, can be administered by various routes of administration. Each agent can be tested over a wide-range of dosages to determine the optimal drug level for each therapeutic agent alone, or in the specific combination thereof, to elicit the maximal response. For these studies, it is preferred to use treatment groups consisting of at least 6 animals per group. Each study is best performed in away wherein the effects of the combination treatment group are determined at the same time as the individual components are evaluated. Although drug effects may be observed with acute administration, it is preferable to observe responses in a chronic setting. The long-term study is of sufficient duration to allow for the full development of compensatory responses to occur and, therefore, the observed effect will most likely depict the actual responses of the test system representing sustained or persistent effects.

Accordingly, a renin inhibitor, or a pharmaceutically acceptable salt thereof, alone or in combination with an ACE inhibitor or an angiotensin II receptor blocker, or in each case, a pharmaceutically acceptable salt thereof, can be tested for its effects on bradykinin levels in the double transgenic rats expressing human renin and human angiotensinogen (dTGR). For example, animals may be treated with aliskiren (1 mg/kg/day-30 mg/kg/day) before the development of disease (prevention design) or after developing the disease (treatment design).

Similarly, a renin inhibitor, or a pharmaceutically acceptable salt thereof, alone or in combination with an ACE inhibitor or an angiotensin II receptor blocker, or in each case, a pharmaceutically acceptable salt thereof, may be tested for its effects on bradykinin levels in Ren-2 transgenic rats, expressing the mouse ren-2 (renin) gene. These rats can be made diabetic by injection with streptozotocin and diastolic dysfunction can be induced by ligating (tying off) a coronary artery to induce a myocardial infarction. Over the ensuing ˜1 month cardiac fibrosis and diastolic dysfunction develop. For example, animals may be treated with aliskiren (1 mg/kg/day-60 mg/kg/day) before the development of the disease (prevention design) or after developing the disease (treatment design).

As an example, diabetes is induced in 6 week old female heterozygous transgenic (mRen-2)27 rats by administration of 55 mg/kg streptozotocin diluted in 0.1 citrate buffer, pH 4.5 by tail vein injection. Non-diabetic rats are injected with citrate buffer alone [Campbell D J, Kelly D J, Wilkinson-Berka J L, Cooper M E, Skinner S L. Increased bradykinin and “normal” angiotensin peptide levels in diabetic Sprague-Dawley and transgenic (mRen-2)27 rats. Kidney Int 1999; 56:211-221.1. Diabetic and non-diabetic rats are randomized to receive either no drug or aliskiren (10 mg/kg/day by osmotic minipump) from 6-7 weeks of age for a duration of 16 weeks. The minipumps are changed every 2 weeks and the amount of aliskiren in the minipumps is adjusted according to the body weight of the rats. Diabetic rats are injected subcutaneously with 2-4 units insulin (Ultratard, Novo Nordisk, Denmark).every second day (3 times a week). If rats have continuously elevated blood glucose levels (>33.3 mmol/L) over a couple of weeks, they are injected with 3 units every day. When glucose levels decrease to 33.3 mmol/L they are administered 2-4 units insulin every second day. After 16 weeks drug treatment, rats are anesthetized with ketamine (75 mg/kg) and xylazine (10 mg/kg) in combination, and blood and tissue is collected for peptide assay. Peptides are measured as previously described [Lawrence A C, Evin G, Kladis A, Campbell D J. An alternative strategy for the radioimmunoassay of angiotensin peptides using amino-terminal-directed antisera: measurement of eight angiotensin peptides in human plasma. J Hypertens 1990; 8:715-724; Campbell D J, Lawrence A C, Towrie A, Kladis A, Valentijn A J. Differential regulation of angiotensin peptide levels in plasma and kidney of the rat. Hypertension 1991; 18:763-773; Campbell D J, Kladis A, Duncan A-M. Nephrectomy, converting enzyme inhibition and angiotensin peptides. Hypertension 1993; 22:513-522; Campbell D J, Kladis A, Duncan A-M. Bradykinin peptides in kidney, blood, and other tissues of the rat. Hypertension 1993; 21:155-165; Campbell D J, Kladis A, Duncan A-M. Effects of converting enzyme inhibitors on angiotensin and bradykinin peptides. Hypertension 1994; 23:439-449; Campbell D J, Rong P, Kladis A, Rees B, Skinner S L. Angiotensin and bradykinin peptides in the TGR(mRen-2)27 rat. Hypertension 1995; 25:1014-1020]. Only the left kidney is collected for peptide assay. The heart refers to the cardiac ventricles (both right and left ventricle). The Ang I levels in heart are below the limit of detection of the assay.

There are 4 groups of rats:
1. Non-diabetic control.
2. Non-diabetic administered aliskiren 10 mg/kg/day.
3. Diabetic control.
4. Diabetic administered aliskiren 10 mg/kg/day.

### Effect of Diabetes: Bradykinin Peptides

Diabetes has been found to increase BK-(1-9) level in kidney, but does not affect BK-(1-9) level in blood, heart, lung, or brain. Diabetes does not affect BK-(1-7) level or BK-(1-7)/(1-9) ratio in blood or any tissue. BK-(1-9) is an abbreviation for bradykinin-(1-9). Bradykinin-(1-9) is understood to represent the amino acids 1-9 of the bradykinin sequence, which in this case is the complete sequence of bradykinin. BK-(1-7) or Bradykinin-(1-7) is a truncated form of bradykinin and lacks the two C-terminal amino acids.

### Effect of Aliskiren: Bradykinin Peptides

Aliskiren has been found to increase both BK-(1-7) and BK-(1-9) levels in heart of both non-diabetic and diabetic rats, with no change in BK-(1-7)/(1-9) ratio. Aliskiren does not affect bradykinin peptides in blood, kidney, lung or brain, although it produces an increase in BK-(1-7)/(1-9) ratio in blood of diabetic, but not non-diabetic, rats.

### Effect of Aliskiren: Bradykinin Peptide Levels

The increase in BK-(1-7) and BK-(1-9) levels in heart of both non-diabetic and diabetic rats is of interest, and indicates either increased bradykinin peptide formation in this tissue or reduced BK-(1-7) and BK-(1-9) metabolism.

The anti-hypertrophic and cardio-protective actions of the kallikrein kinin system and of bradykinin in both non-diabetic and diabetic animal models are well documented and this suggests for human therapy that they contribute to the therapeutic benefits of aliskiren therapy by e.g. protecting the heart from ischemic, oxidative, inflammatory and/or hemodynamic insult. This tissue-specific effect of aliskiren on cardiac bradykinin peptide levels makes it unlikely that aliskiren will cause angioneurotic edema, either alone or in combination with e.g. an ACE inhibitor.

Summary data: Effect of aliskiren on peptide levels

| | | | | | | Ang II/ |
|---|---|---|---|---|---|---|
| | | | BK-(1-7)/ | Peptide | | Ang I |
| Tissue | BK-(1-7) | BK-(1-9) | (1-9) ratio | Ang II | Ang I | ratio |
| Control | | | | | | |
| rats | | | | | | |
| Blood | [symbol] | [symbol] | [symbol] | [symbol] | [symbol] | [symbol] |
| Kidney | [symbol] | [symbol] | [symbol] | [symbol] | [symbol] | [symbol] |
| Heart | [symbol] | [symbol] | [symbol] | [symbol] | ND | ND |
| Lung | [symbol] | [symbol] | [symbol] | [symbol] | [symbol] | [symbol] |
| Brain | [symbol] | [symbol] | [symbol] | [symbol] | [symbol] | [symbol] |
| Diabetic | | | | | | |
| rats | | | | | | |
| Blood | [symbol] | [symbol] | [symbol] | [symbol] | [symbol] | [symbol] |
| Kidney | [symbol] | [symbol] | [symbol] | [symbol] | [symbol] | [symbol] |
| Heart | [symbol] | [symbol] | [symbol] | [symbol] | ND | ND |
| Lung | [symbol] | [symbol] | [symbol] | [symbol] | [symbol] | [symbol] |
| Brain | [symbol] | [symbol] | [symbol] | [symbol] | [symbol] | [symbol] |

Heart bradykinin peptides

| Group | Mean ± SEM | Dunnett's P |
|---|---|---|
| BK-(1-7) (fmol/g) | | |
| Control (n = 10) | 7.9 ± 1.0 | |
| Control + Aliskiren (n = 10) | 19.2 ± 2.5 | <0.01 |
| Diabetic (n = 11) | 7.1 ± 0.9 | |
| Diabetic + Aliskiren (n = 10) | 13.9 ± 1.5 | <0.01 |
| BK-(1-9) (fmol/g) | | |
| Control (n = 10) | 4.9 ± 0.8 | |
| Control + Aliskiren (n = 10) | 10.6 ± 1.2 | <0.01 |
| Diabetic (n = 11) | 4.1 ± 0.7 | |
| Diabetic + Aliskiren (n = 10) | 6.4 ± 0.7 | <0.05 |
| BK-(1-7)/(1-9) ratio (mol/mol) | | |
| Control (n = 10) | 1.90 ± 0.27 | |
| Control + Aliskiren (n = 10) | 1.88 ± 0.18 | |
| Diabetic (n = 11) | 2.16 ± 0.49 | |
| Diabetic + Aliskiren (n = 10) | 2.47 ± 0.37 | |

### Effect of Diabetes (No Drug Therapy)

Diabetes did not affect either the BK-(1-7) or BK-(1-9) levels, or the BK-(1-7)/(1-9) ratio.

### Effect of Drug Therapy in Control Rats

In control rats, aliskiren increased heart BK-(1-7) and BK-(1-9) levels. Drug therapy did not affect the BK-(1-7)/(1-9) ratio.

### Effect of Drug Therapy in Diabetic Rats

In diabetic rats, aliskiren increased heart BK-(1-7) and BK-(1-9) levels. Drug therapy did not affect the BK-(1-7)/(1-9) ratio.

Furthermore, it has been found that, a combination of a renin inhibitor, e.g., aliskiren, especially in the form of the hemi-fumarate salt thereof, and an ACE inhibitor, e.g., benazepril or enalapril, or an angiotensin II receptor blocker, e.g., valsartan, or in each case, a pharmaceutically acceptable salt thereof, achieves also a therapeutic effect.

It can be shown that combination therapy with a renin inhibitor, e.g., aliskiren, especially in the form of the hemi-fumarate salt thereof, and an ACE inhibitor, e.g., benazepril or enalapril, or an angiotensin II receptor blocker, e.g., valsartan, or in each case, a pharmaceutically acceptable salt thereof, results in an effective therapy for the prevention of, delay progression to overt to, or the treatment of diseases modulated by an increase in tissue bradykinin levels.

The invention furthermore relates to the use of a renin inhibitor, e.g., aliskiren, alone or in combination with an ACE inhibitor, e.g., benazepril or enalapril, or an angiotensin II receptor blocker, e.g., valsartan, or in each case, a pharmaceutically acceptable salt thereof, for the manufacture of a medicament for the prevention of, delay progression to overt to, or the treatment of diseases modulated by an increase in tissue bradykinin levels.

Accordingly, another embodiment of the present invention relates to the use of a renin inhibitor, e.g., aliskiren, alone or in combination with an ACE inhibitor, e.g., benazepril or enalapril, or an angiotensin II receptor blocker, e.g., valsartan, or in each case, a pharmaceutically acceptable salt thereof, for the manufacture of a medicament for the prevention of, delay progression to overt to, or the treatment diseases modulated by an increase in tissue bradykinin levels.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative of certain aspects of the present invention and are not a limitation of the scope of the present invention in any way.

### FORMULATION EXAMPLE 1

Composition of aliskiren 150 mg (free base) uncoated tablets in mg/unit.

| | Roller | | | |
|---|---|---|---|---|
| | compacted | Dosage | Dosage | Dosage |
| Component | tablet | form 1 | form 2 | form 3 |
| Aliskiren | 165.750 | 165.750 | 165.750 | 165.750 |
| hemi-fumarate | | | | |
| Microcrystalline | 220.650 | 84.750 | 72.250 | 107.250 |
| cellulose | | | | |
| Polyvinylpyrrolidon | — | — | 12.000 | 12.000 |
| K 30 | | | | |
| Crospovidone | 84.000 | 45.000 | 44.000 | 48.200 |
| Aerosil 200 | 4.800 | 1.500 | 1.500 | 1.800 |
| Magnesium stearate | 4.800 | 3.000 | 4.500 | 5.000 |
| Total weight | 480.000 | 300.000 | 300.000 | 340.000 |

Composition of aliskiren 150 mg (free base) uncoated tablets in % by weight.

| | Roller | | | |
|---|---|---|---|---|
| | compacted | Dosage | Dosage | Dosage |
| Component | tablet | form 1 | form 2 | form 3 |
| Aliskiren | 34.53 | 55.25 | 55.25 | 48.75 |
| hemi-fumarate | | | | |
| Microcrystalline | 45.97 | 28.25 | 24.08 | 31.545 |
| cellulose | | | | |
| Polyvinylpyrrolidon | — | — | 4 | 3.53 |
| K 30 | | | | |
| Crospovidone | 17.5 | 15 | 14.67 | 14.175 |
| Aerosil 200 | 1 | 0.5 | 0.5 | 0.53 |
| Magnesium stearate | 1 | 1 | 1.5 | 1.47 |
| Total % | 100.00 | 100.00 | 100.00 | 100.00 |

Composition of aliskiren 150 mg (free base) uncoated tablets in mg/unit (divided into inner/outer phase).

| | | Roller | | | |
|---|---|---|---|---|---|
| | | compacted | Dosage | Dosage | Dosage |
| | Component | tablet | form 1 | form 2 | form 3 |
| Inner | Aliskiren | 165.75 | 165.75 | 165.75 | 165.75 |
| Phase | hemi-fumarate | | | | |
| | Microcrystalline | 220.65 | 84.75 | 72.25 | 90.25 |
| | cellulose | | | | |
| | Polyvinylpyrrolidon | — | — | 12.00 | 12.00 |
| | K 30 | | | | |
| | Crospovidone | 36.00 | — | — | 14.20 |
| | Aerosil 200 | — | — | — | — |
| | Magnesium stearate | 2.40 | — | — | — |
| Outer | Crospovidone | 48.00 | 45.00 | 44.00 | 34.00 |
| phase | Microcrystalline | — | — | — | 17.00 |
| | cellulose | | | | |
| | Aerosil 200 | 4.80 | 1.50 | 1.50 | 1.80 |
| | Magnesium stearate | 2.40 | 3.00 | 4.50 | 5.00 |
| | Total weight | 480.00 | 300.00 | 300.00 | 340.00 |

Composition of aliskiren 150 mg (free base) uncoated tablets in % by weight (divided into inner/outer phase).

| | | Roller | | | |
|---|---|---|---|---|---|
| | | compacted | Dosage | Dosage | Dosage |
| | Component | tablet | form 1 | form 2 | form 3 |
| Inner | Aliskiren | 34.53 | 55.25 | 55.25 | 48.75 |
| Phase | hemi-fumarate | | | | |
| | Microcrystalline | 45.97 | 28.25 | 24.08 | 26.545 |
| | cellulose | | | | |
| | Polyvinylpyrrolidon | — | — | 4 | 3.530 |
| | K 30 | | | | |
| | Crospovidone | 7.5 | — | — | 4.175 |
| | Aerosil 200 | — | — | — | — |
| | Magnesium stearate | 0.5 | — | — | — |
| Outer | Crospovidone | 10 | 15 | 14.67 | 10 |
| phase | Microcrystalline | — | — | — | 5 |
| | cellulose | | | | |
| | Aerosil 200 | 1 | 0.5 | 0.5 | 0.53 |
| | Magnesium stearate | 0.5 | 1 | 1.5 | 1.47 |
| | Total % | 100.00 | 100.00 | 100.00 | 100.00 |

### FORMULATION EXAMPLE 2

Composition of aliskiren (dosage form 3) film-coated tablets in mg/unit.

| | Dosage form 3/Strength | | |
|---|---|---|---|
| | 75 mg | 150 mg | 300 mg |
| Component | (free base) | (free base) | (free base) |
| Aliskiren | 82.875 | 165.750 | 331.500 |
| hemi-fumarate | | | |
| Microcrystalline | 53.625 | 107.250 | 214.500 |
| cellulose | | | |
| Polyvinylpyrrolidon | 6.000 | 12.000 | 24.000 |
| K 30 | | | |
| Crospovidone | 24.100 | 48.200 | 96.400 |
| Aerosil 200 | 0.900 | 1.800 | 3.600 |
| Magnesium stearate | 2.500 | 5.000 | 10.000 |
| Total tablet weight | 170.000 | 340.000 | 680.000 |
| Opadry premix white | 9.946 | 16.711 | 23.9616 |
| Opadry premix red | 0.024 | 0.238 | 1.8382 |
| Opadry premix black | 0.030 | 0.051 | 0.2002 |
| Total fim-coated | 180.000 | 357.000 | 706.000 |
| tablet weight | | | |

The dosage forms 1, 2 and 3 may be prepared, e.g., as follows:
1) mixing the active ingredient and additives and granulating said components with a granulation liquid;
2) drying a resulting granulate;
3) mixing the dried granulate with outer phase excipients;
4) compressing a resulting mixture to form a solid oral dosage as a core tablet; and
5) optionally coating a resulting core tablet to give a film-coated tablet.

The granulation liquid can be ethanol, a mixture of ethanol and water, a mixture of ethanol, water and isopropanol, or a solution of polyvinylpyrrolidones (PVP) in the before mentioned mixtures. A preferred mixture of ethanol and water ranges from about 50/50 to about 99/1 (% w/w), most preferrably it is about 94/6 (% w/w). A preferred mixture of ethanol, water and isopropanol ranges from about 45/45/5 to about 98/1/1 (% w/w/w), most preferably from about 88.5/5.5/6.0 to about 91.5/4.5/4.0 (% w/w/w). A preferred concentration of PVP in the above named mixtures ranges from about 5 to about 30% by weight, preferably from about 15 to about 25%, more preferably from about 16 to about 22%.

Attention is drawn to the numerous known methods of granulating, drying and mixing employed in the art, e.g., spray granulation in a fluidized bed, wet granulation in a high-shear mixer, melt granulation, drying in a fluidized-bed dryer, mixing in a free-fall or tumble blender, compressing into tablets on a single-punch or rotary tablet press.

The manufacturing of the granulate can be performed on standard equipment suitable for organic granulation processes. The manufacturing of the final blend and the compression of tablets can also be performed on standard equipment.

For example, step (1) may be carried out by a high-shear granulator, e.g., Collette Gral; step (2) may be conducted in a fluid-bed dryer; step (3) may be carried out by a free-fall mixer (e.g. container blender, tumble blender); and step (4) may be carried out using a dry compression method, e.g., a rotary tablet press.

### FORMULATION EXAMPLE 3

### Film-Coated Tablets)

| | Composition | |
|---|---|---|
| Components | Per Unit (mg) | Standards |
| Granulation | | |
| Valsartan [=active ingredient] | 80.00 | |
| Microcrystalline cellulose/ | 54.00 | NF, Ph. Eur |
| Avicel PH 102 | | |
| Crospovidone | 20.00 | NF, Ph. Eur |
| Colloidal anhydrous silica/ | 0.75 | Ph. Eur/NF |
| colloidal silicon dioxide/ | | |
| Aerosil 200 | | |
| Magnesium stearate | 2.5 | NF, Ph. Eur |
| Blending | | |
| Colloidal anhydrous silica/ | 0.75 | Ph. Eur/NF |
| colloidal silicon dioxide/ | | |
| Aerosil 200 | | |
| Magnesium stearate | 2.00 | NF, Ph. Eur |
| Coating | | |
| Purified water *⁾ | — | |
| DIOLACK pale red 00F34899 | 7.00 | |
| Total tablet mass | 167.00 | |

*⁾ Removed during processing.

The film-coated tablets may be manufactured, e.g., as follows:

A mixture of valsartan, microcrystalline cellulose, crospovidone, part of the colloidal anhydrous silica/colloidal silicon dioxide/Aerosile 200, silicon dioxide and magnesium stearate is premixed in a diffusion mixer and then sieve through a screening mill. The resulting mixture is again pre-mixed in a diffusion mixer, compacted in a roller compactor and then sieve through a screening mill. To the resulting mixture, the rest of the colloidal anhydrous silica/colloidal silicon dioxide/Aerosile 200 are added and the final blend is made in a diffusion mixer. The whole mixture is compressed in a rotary tabletting machine and the tablets are coated with a film by using Diolack pale red in a perforated pan.

### FORMULATION EXAMPLE 4

### Film-Coated Tablets

| | Composition | |
|---|---|---|
| Components | Per Unit (mg) | Standards |
| Granulation | | |
| Valsartan [=active ingredient] | 160.00 | |
| Microcrystalline cellulose/ | 108.00 | NF, Ph. Eur |
| Avicel PH 102 | | |
| Crospovidone | 40.00 | NF, Ph. Eur |
| Colloidal anhydrous silica/ | 1.50 | Ph. Eur/NF |
| colloidal silicon dioxide/ | | |
| Aerosil 200 | | |
| Magnesium stearate | 5.00 | NF, Ph. Eur |
| Blending | | |
| Colloidal anhydrous silica/ | 1.50 | Ph. Eur/NF |
| colloidal silicon dioxide/ | | |
| Aerosil 200 | | |
| Magnesium stearate | 4.00 | NF, Ph. Eur |
| Coating | | |
| Opadry Light Brown 00F33172 | 10.00 | |
| Total tablet mass | 330.00 | |

The film-coated tablets are manufactured, e.g., as described in Example 3.

### FORMULATION EXAMPLE 5

### Film-Coated Tablets

| | Composition | |
|---|---|---|
| Components | Per Unit (mg) | Standards |
| Core: internal phase | | |
| Valsartan [=active ingredient] | 40.00 | |
| Silica, colloidal anhydrous | 1.00 | Ph. Eur, USP/NF |
| (Colloidal silicon dioxide) | | |
| [=Glidant] | | |
| Magnesium stearate | 2.00 | USP/NF |
| [=Lubricant] | | |
| Crospovidone | 20.00 | Ph. Eur |
| [Disintegrant] | | |
| Microcrystalline cellulose | 124.00 | USP/NF |
| [=Binding agent] | | |
| External phase | | |
| Silica, colloidal anhydrous, | 1.00 | Ph. Eur, USP/NF |
| (Colloidal silicon dioxide) | | |
| [=Glidant] | | |
| Magnesium stearate | 2.00 | USP/NF |
| [Lubricant] | | |
| Film coating | | |
| Opadry ® brown OOF 16711*⁾ | 9.40 | |
| Purified Water**⁾ | — | |
| Total tablet mass | 199.44 | |

*⁾The composition of the Opadry ® brown OOF16711 coloring agent is tabulated below.
**⁾Removed during processing.

### Opadry® Composition:

| | Approximate % |
|---|---|
| Ingredient | Composition |
| Iron oxide, black (C.I. No. 77499, E 172) | 0.50 |
| Iron oxide, brown (C.I. No. 77499, E 172 | 0.50 |
| Iron oxide, red (C.I. No. 77491, E 172) | 0.50 |
| Iron oxide, yellow (C.I. No. 77492, E 172) | 0.50 |
| Macrogolum (Ph. Eur) | 4.00 |
| Titanium dioxide (C.I. No. 77891, E 171) | 14.00 |
| Hypromellose (Ph. Eur) | 80.00 |

The film-coated tablets are manufactured, e.g., as described in Example 3.

### FORMULATION EXAMPLE 6

### Capsules

| | Composition |
|---|---|
| | Per Unit (mg) |
| Components | |
| Valsartan [=active ingredient] | 80.00 |
| Microcrystalline cellulose | 25.10 |
| Crospovidone | 13.00 |
| Povidone | 12.50 |
| Magnesium stearate | 1.30 |
| Sodium lauryl sulphate | 0.60 |
| Shell | |
| Iron oxide, red (C.I. No. 77491, EC No. E 172) | 0.123 |
| Iron oxide, yellow (C.I. No. 77492, EC No. E 172) | 0.123 |
| Iron oxide, black (C.I. No. 77499, EC No. E 172) | 0.245 |
| Titanium dioxide | 1.540 |
| Gelatin | 74.969 |
| Total mass | 209.50 |

The capsules may be manufactured, e.g., as follows:

### Granulation/Drying:

Valsartan and microcrystallin cellulose are spray-granulated in a fluidized bed granulator with a granulating solution consisting of povidone and sodium lauryl sulphate dissolved in purified water. The granulate obtained is dried in a fluidized bed dryer.

### Milling/Blending:

The dried granulate is milled together with crospovidone and magnesium stearate. The mass is then blended in a conical srew type mixer for approximately 10 minutes.

### Encapsulation:

The empty hard gelatin capsules are filled with the blended bulk granules under controlled temperature and humidity conditions. The filed capsules are dedusted, visually inspected, weightchecked and quarantined until by Quality assurance department.

### FORMULATION EXAMPLE 7

### Capsules

| | Composition |
|---|---|
| | Per Unit (mg) |
| Components | |
| Valsartan [=active ingredient] | 160.00 |
| Microcrystalline cellulose | 50.20 |
| Crospovidone | 26.00 |
| Povidone | 25.00 |
| Magnesium stearate | 2.60 |
| Sodium lauryl sulphate | 1.20 |
| Shell | |
| Iron oxide, red (C.I. No. 77491, EC No. E 172) | 0.123 |
| Iron oxide, yellow (C.I. No. 77492, EC No. E 172) | 0.123 |
| Iron oxide, black (C.I. No. 77499, EC No. E 172) | 0.245 |
| Titanium dioxide | 1.540 |
| Gelatin | 74.969 |
| Total mass | 342.00 |

The capsules are manufactured, e.g., as described in Example 6.

### FORMULATION EXAMPLE 8

### Hard Gelatine Capsules

| | Composition |
|---|---|
| Components | Per Unit (mg) |
| Valsartan [=active ingredient] | 80.00 |
| Sodium laurylsulphate | 0.60 |
| Magnesium stearate | 1.30 |
| Povidone | 12.50 |
| Crospovidone | 13.00 |
| Microcrystalline cellulose | 21.10 |
| Total mass | 130.00 |

### FORMULATION EXAMPLE 9

### Hard Gelatin Capsules

| | Composition |
|---|---|
| Components | Per Unit (mg) |
| Valsartan [=active ingredient] | 80.00 |
| Microcrystalline cellulose | 110.00 |
| Povidone K30 | 45.20 |
| Sodium laurylsulphate | 1.20 |
| Magnesium stearate | 2.60 |
| Crospovidone | 26.00 |
| Total mass | 265.00 |

Components (1) and (2) are granulated with a solution of components (3) and (4) in water. The components (5) and (6) are added to the dry granulate and the mixture is filled into size 1 hard gelatin capsules.

All publications and patents mentioned herein are incorporate by reference in their entirety as if set forth in full herein.

1. A method for the prevention of, delay of progression to overt to, or the treatment of diseases modulated by an increase in tissue bradykinin levels which method comprises administering to a warm-blooded animal a therapeutically effective amount of a renin inhibitor, or a pharmaceutically acceptable salt thereof.

2. The method according to claim 1, wherein a renin inhibitor is selected from the group consisting of a compound of formula (III) 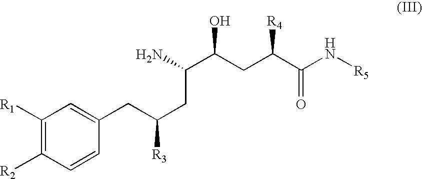
wherein R₁ is halogen, C₁₋₆halogenalkyl, C₁₋₆alkoxy-C₁₋₆alkyloxy or C₁₋₆alkoxy-C₁₋₆alkyl; R₂ is halogen, C₁alkyl or C₁₋₄alkoxy; R₃ and R₄ are independently branched C₃₋₆alkyl; and R₅ is cycloalkyl, C₁₋₆alkyl, C₁₋₆hydroxyalkyl, C₁₋₆alkoxy-C₁₋₆alkyl, C₁₋₆alkanoyloxy-C₁₋₆alkyl, C₁₋₆aminoalkyl, C₁₋₆alkylamino-C₁₋₆alkyl, C₁₋₆dialkylamino-C₁₋₆alkyl, C₁₋₆alkanoylamino-C₁₋₆alkyl, HO(O)C-C₁₋₆alkyl, C₁₋₆alkyl-O—(O)C-C₁₋₆alkyl, H₂N—C(O)—C₁₋₆alkyl, C₁₋₆alkyl-HN—C(O)—C₁₋₆alkyl or (C₁₋₆alkyl)₂N—C(O)—C₁₋₆alkyl; or in each case a pharmaceutically acceptable salt thereof.

3. The method according to claim 2, wherein the renin inhibitor is a compound of formula (III) having the formula 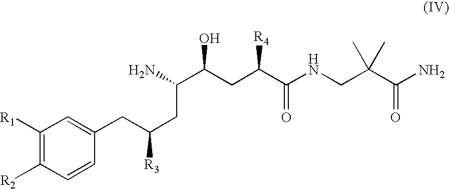
wherein R₁ is 3-methoxypropyloxy; R₂ is methoxy; and R₃ and R₄ are isopropyl; or a pharmaceutically acceptable salt thereof.

4. The method according to claim 3, wherein the compound of formula (IV) is in the form of the hemi-fumarate salt thereof.

5. The method according to claim 1, wherein the disease is selected from ischaemic heart disease and ischaemic heart damage.

6. A pharmaceutical composition comprising a renin inhibitor, or a pharmaceutically acceptable salt thereof, for the prevention of, delay of progression to, or the treatment of diseases modulated by an increase in tissue bradykinin levels.

7. The pharmaceutical composition according to claim 6, wherein the renin inhibitor is a compound of formula (III) 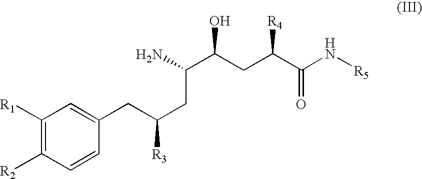
wherein R₁ is halogen, C₁₋₆halogenalkyl, C₁₋₆alkoxy-C₁₋₆alkyloxy or C₁₋₆alkoxy-C₁₋₆alkyl; R₂ is halogen, C₁₋₄alkyl or C₁₋₄alkoxy; R₃ and R₄ are independently branched C₃₋₆alkyl; and R₅ is cycloalkyl, C₁₋₆alkyl, C₁₋₆hydroxyalkyl, C₁₋₆alkoxy-C₁₋₆alkyl, C₁₋₆alkanoyloxy-C₁₋₆alkyl, C₁₋₆aminoalkyl, C₁₋₆alkylamino-C₁₋₆alkyl, C₁₋₆alkanoylamino-C₁₋₆alkyl, HO(O)C-C₁₋₆alkyl, C₁₋₆alkyl-O—(O)C-C₁₋₆alkyl, H₂N—C(O)—C₁₋₆alkyl, C₁₋₆alkyl-HN—C(O)—C₁₋₆alkyl or (C₁₋₆alkyl)₂N—C(O)—C₁₋₆alkyl; or in each case a pharmaceutically acceptable salt thereof.

8. The pharmaceutical composition according to claim 7, wherein the renin inhibitor is a compound of formula (III) having the formula 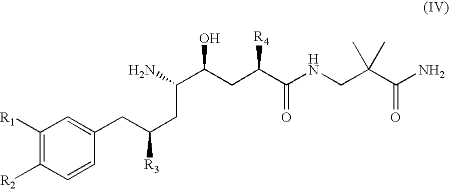
wherein R₁ is 3-methoxypropyloxy; R₂ is methoxy; and R₃ and R₄ are isopropyl; or a pharmaceutically acceptable salt thereof.

9. The pharmaceutical composition according to claim 8, wherein the compound of formula (IV) is in the form of the hemi-fumarate salt thereof.

10. The pharmaceutical composition according to claim 6, wherein the disease is selected from ischaemic heart disease and ischaemic heart damage.

11. Use of a renin inhibitor for the manufacture of a medicament for the prevention of, delay progression to, or the treatment of diseases modulated by an increase in tissue bradykinin levels.

12. The use according to claim 11 wherein a renin inhibitor is selected from a compound of the formula 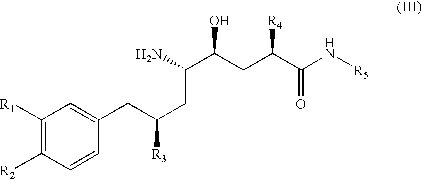
wherein R₁ is halogen, C₁₋₆halogenalkyl, C₁₋₆alkoxy-C₁₋₆alkyloxy or C₁₋₆alkoxy-C₁₋₆alkyl; R₂ is halogen, C₁₋₄alkyl or C₁₋₄alkoxy; R₃ and R₄ are independently branched C₃₋₆alkyl; and R₅ is cycloalkyl, C₁₋₆alkyl, C₁₋₆hydroxyalkyl, C₁₋₆alkoxy-C₁₋₆alkyl, C₁₋₆alkanoyloxy-alkyl, C₁₋₆aminoalkyl, C₁₋₆dialkylamino-C₁₋₆alkyl, C₁₋₆alkanoylamino-HO(O)C-C₁₋₆alkyl, C₁₋₆alkyl-O—(O)C-C₁₋₆alkyl, H₂N—C(O)—C₁₋₆alkyl, C₁₋₆alkyl-HN—C(O)—C₁₋₆alkyl or (C₁₋₆alkyl)₂N—C(O)—C₁₋₆alkyl; or in each case a pharmaceutically acceptable salt thereof.

13. The use according to claim 12, wherein a compound of formula (III) has the formula 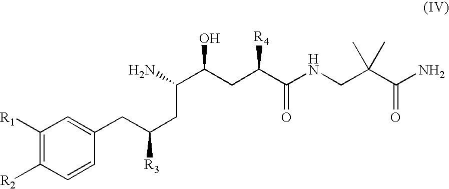
wherein R₁ is 3-methoxypropyloxy; R₂ is methoxy; and R₃ and R₄ are isopropyl; or a pharmaceutically acceptable salt thereof.

14. The use according to claim 13, wherein the compound of formula (IV) is in the form of the hemi-fumarate salt thereof.

15. The use according to claim 11, wherein the disease is selected from ischaemic heart disease and ischaemic heart damage.
